(19) United States
(12) Patent Application Publication
(10) Pub. No.: US 2024/0326051 A1
(43) Pub. Date: Oct. 3, 2024

(54) Microfluidic Devices

(71) Applicant: Donaldson Company, Inc., Bloomington, MN (US)

(72) Inventors: Jacob A. Nelson, Bloomington, MN (US); Connor J. Colling, Minneapolis, MN (US); Bradly G. Hauser, Minneapolis, MN (US); Davis B. Moravec, Burnsville, MN (US)

(21) Appl. No.: 18/621,745

(22) Filed: Mar. 29, 2024

Related U.S. Application Data
(60) Provisional application No. 63/455,886, filed on Mar. 30, 2023.

(51) Int. Cl. B01L 3/00

(52) U.S. Cl. CPC B01L 3/502761; B01L 2200/027; B01L 2200/0647; B01L 2300/0864

(57) ABSTRACT

A separation assembly has a microfluidic separation element having a substrate layer. The microfluidic separation element defines an element inlet, a first element outlet, and a second element outlet. The first element outlet is defined by the substrate layer. A permeate draining layer abuts the substrate layer. The permeate draining layer is downstream of the first element outlet.

### PRIORITY

This application claims the benefit of U.S. Provisional Application No. 63/455,886, filed Mar. 30, 2023, the disclosure of which is incorporated by reference herein in its entirety.

### TECHNOLOGICAL FIELD

The present disclosure is generally related to microfluidic devices. More particularly, the present disclosure is related to microfluidic devices as a particle separator.

### SUMMARY

Some embodiments of the technology disclosed herein relate to a separation assembly that has a microfluidic separation element having a substrate layer. The microfluidic separation element defines an element inlet, a plurality of first element outlets, and a second element outlet. The plurality of first element outlets are defined by the substrate layer. A permeate draining layer abuts the substrate layer. The permeate draining layer is downstream of the plurality of first element outlets.

In some such embodiments, the permeate draining layer extends laterally across the substrate layer. Additionally or alternatively, the separation assembly is a component of a cell retention device of a perfusion bioreactor. Additionally or alternatively, the microfluidic separation element is a hydrodynamic separator element. Additionally or alternatively, the permeate draining layer is defined by a mesh material. Additionally or alternatively, the permeate draining layer is a material layer defining a microchannel downstream of the plurality of first layer outlets. Additionally or alternatively, the separation assembly further has a first microfluidic channel defining a channel inlet downstream of the element inlet and a channel outlet having a first channel outlet upstream of a first element outlet of the plurality of first element outlets and a second channel outlet upstream of the second element outlet. Additionally or alternatively, the first microfluidic channel is curved.

Additionally or alternatively, the separation assembly has a plurality of microfluidic channels having the first microfluidic channel. Each microfluidic channel defines a channel inlet downstream of the element inlet and a first channel outlet upstream of a first element outlet of the plurality of first element outlets and a second channel outlet upstream of the second element outlet. Additionally or alternatively, each of the plurality of microfluidic channels is nested with an adjacent microfluidic channel. Additionally or alternatively, the plurality of microfluidic channels has at least 6 microfluidic channels. Additionally or alternatively, each microfluidic channel is within 12 mm, 10 mm, 5 mm, or 3 mm of an adjacent microfluidic channel. Additionally or alternatively, each microfluidic channel is curved. Additionally or alternatively, each microfluidic channel defines two curves in opposite directions. Additionally or alternatively, each microfluidic channel has an inner wall and an outer wall, and each microfluidic channel is configured to concentrate particles in a liquid stream towards the inner wall. Additionally or alternatively, the length of each of the plurality of microfluidic channels defines an arc of less than or equal to 270°.

Some embodiments of the current technology relate to a system having a plurality of separation assemblies consistent with those described above, where the separation assemblies are arranged in a stacked configuration. A system inlet is in direct fluid communication with each element inlet. The separation assemblies are arranged to operate in parallel.

Additionally or alternatively, the length of each of the plurality of microfluidic channels defines an arc of less than or equal to 200°. Additionally or alternatively, the microfluidic separation element is configured to separate particles ranging from 10-20 microns from a liquid stream. Additionally or alternatively, the microfluidic separation element is configured to separate one or both of fish cells and avian cells in a liquid stream. Additionally or alternatively, the microfluidic separation element is configured to separate one or both of mammalian cells and insect cells in a liquid stream.

Some embodiments of the technology disclosed herein relate to a hydrodynamic separator element has a substrate layer defines a layer inlet, a plurality of first layer outlets and a second layer outlet, an outlet flow path upstream of the second layer outlet, and a plurality of curved microfluidic channels arranged to operate in parallel. Each microfluidic channel defines a channel inlet downstream of the layer inlet, a first channel outlet upstream of a first layer outlet of the plurality of first layer outlets, and a second channel outlet upstream of the outlet flow path. The substrate layer is non-permeable. Each of the plurality of microfluidic channels is nested with an adjacent microfluidic channel.

In some such embodiments, the length of each of the plurality of curved microfluidic channels defines an arc of less than or equal to 270°. Additionally or alternatively, the length of each of the plurality of curved microfluidic channels defines an arc of less than or equal to 200°. Additionally or alternatively, each microfluidic channel defines two curves in opposite directions. Additionally or alternatively, the plurality of curved microfluidic channels is at least 6 microfluidic channels. Additionally or alternatively, each microfluidic channel is within 10 mm, 5 mm, or 3 mm of an adjacent microfluidic channel. Additionally or alternatively, the hydrodynamic separator element further has a permeate draining layer abutting the substrate layer. The permeate draining layer is downstream of the first layer outlets.

Additionally or alternatively, the permeate draining layer extends laterally across the substrate layer. Additionally or alternatively, the permeate draining layer is defined by a mesh material. Additionally or alternatively, the permeate draining layer is a material layer defining a microchannel downstream of the plurality of first layer outlets. Additionally or alternatively, the hydrodynamic separator element is a component of a cell retention device of a perfusion bioreactor. Additionally or alternatively, each microfluidic channel has an inner wall and an outer wall, and each microfluidic channel is configured to concentrate particles in a liquid stream towards the inner wall. Additionally or alternatively, the hydrodynamic separator element is configured to separate particles ranging from 10-20 microns from a liquid stream. Additionally or alternatively, the hydrodynamic separator element is configured to separate one or both of fish cells and avian cells in a liquid stream. Additionally or alternatively, the hydrodynamic separator element is configured to separate one or both of mammalian cells and insect cells in a liquid stream.

Additionally or alternatively, the hydrodynamic separator element has a plurality of the substrate layers in a stacked configuration. A system inlet is in direct fluid communication with each layer inlet, and the plurality of substrate layers are arranged to operate in parallel. Additionally or alternatively, a permeate draining layer abuts each substrate layer, wherein each permeate draining layer is downstream of a corresponding first layer outlet.

Some embodiments of the technology disclosed herein relate to a system has a hydrodynamic separator element having an element inlet, a plurality of first element outlets, a second element outlet, and a plurality of curved microfluidic channels between the element inlet and the first element outlet. Each curved microfluidic channel has an inner wall and an outer wall and the hydrodynamic separator element is configured to concentrate particles along the inner wall. A tangential flow filter has a feed inlet downstream of the plurality of first element outlets, a retentate outlet, a permeate outlet, and a filter media disposed between the feed inlet and the permeate outlet.

In some such embodiments, the system further has a cell culture tank positioned downstream of the tangential flow filter. Additionally or alternatively, the hydrodynamic separator element has a substrate layer defining a layer inlet, a plurality of first layer outlets, and a second layer outlet. The layer inlet is downstream of the element inlet, each first layer outlet is upstream of a first element outlet of the plurality of first element outlets, and the second layer outlet is upstream of the second element outlet. Each of the plurality of curved microfluidic channels extend between the layer inlet and a first layer outlet of the plurality of first layer outlets. Additionally or alternatively, the system further has a permeate draining layer abutting the substrate layer. The permeate draining layer is downstream of the first layer outlets.

Additionally or alternatively, the permeate draining layer extends laterally across the substrate layer. Additionally or alternatively, the hydrodynamic separator element has a plurality of substrate layers in a stacked configuration. Each substrate layer defines a layer inlet, a plurality of first layer outlets and a second layer outlet, and a curved microfluidic channel extending between the layer inlet and a first layer outlet of the plurality of first layer outlets. Each layer inlet is downstream of the element inlet, each first layer outlet is upstream of a first element outlet of the plurality of first element outlets, and each second layer outlet is upstream of the second element outlet. Additionally or alternatively, the system further has a permeate draining layer abutting each substrate layer. The permeate draining layer is downstream of a corresponding first layer outlet. Additionally or alternatively, each permeates draining layer extends laterally across a corresponding substrate layer. Additionally or alternatively, the permeate draining layer is a material layer defining a microchannel downstream of the plurality of first layer outlets. Additionally or alternatively, the permeate draining layer is defined by a mesh material. Additionally or alternatively, each substrate layer defines a plurality of curved microfluidic channels each extending between the layer inlet and a first layer outlet of the plurality of first layer outlets.

Additionally or alternatively, each of the plurality of microfluidic channels is nested with an adjacent microfluidic channel. Additionally or alternatively, each substrate layer has at least 6 microfluidic channels. Additionally or alternatively, each microfluidic channel is within 12 mm, 10 mm, 5 mm, or 3 mm of an adjacent microfluidic channel within each substrate layer. Additionally or alternatively, each microfluidic channel defines two curves in opposite directions. Additionally or alternatively, the length of each of the plurality of curved microfluidic channels defines an arc of less than or equal to 270°.

Additionally or alternatively, the length of each of the plurality of curved microfluidic channels defines an arc of less than or equal to 200°. Additionally or alternatively, the hydrodynamic separator element is configured to separate particles having diameters ranging from 10-20 microns from a liquid stream. Additionally or alternatively, the hydrodynamic separator element is configured to separate one or both of fish cells and avian cells in a liquid stream. Additionally or alternatively, the hydrodynamic separator element is configured to separate one or both of mammalian cells and insect cells in a liquid stream.

The above summary is not intended to describe each embodiment or every implementation. Rather, a more complete understanding of illustrative embodiments will become apparent and appreciated by reference to the following Detailed Description of Exemplary Aspects and claims in view of the accompanying figures of the drawing.

### BRIEF DESCRIPTION OF THE DRAWINGS

The present technology may be more completely understood and appreciated in consideration of the following detailed description of various embodiments in connection with the accompanying drawings.

FIG. 1 is a schematic representation of an example microfluidic device consistent with the technology disclosed herein.

FIG. 2 is a perspective view of a portion of an example microfluidic separator element that can be used in a device of FIG. 1.

FIG. 3 is a detail view of FIG. 2.

FIG. 4 is a cross-sectional view of an example flow channel consistent with the technology disclosed herein.

FIG. 5 is a lateral facing view of a plurality of flow channels consistent with the technology disclosed herein.

FIG. 6 is a perspective cross-sectional view of the portion of the example microfluidic separator element of FIG. 2.

FIG. 7 is a lateral facing view of another example of a plurality of flow channels consistent with the technology disclosed herein

FIG. 8 is a lateral facing view of yet another example of a plurality of flow channels consistent with the technology disclosed herein.

FIG. 9 is a lateral facing view of yet another example of a plurality of flow channels consistent with the technology disclosed herein.

FIG. 10 is a cross-sectional schematic view of an example separator system consistent with the technology disclosed herein.

FIG. 11 Is a schematic view of an example implementation consistent with the technology disclosed herein.

The figures are rendered primarily for clarity and, as a result, are not necessarily drawn to scale. Moreover, various structure/components, including but not limited to fasteners, electrical components (wiring, cables, etc.), and the like, may be shown diagrammatically or removed from some or all of the views to better illustrate aspects of the depicted embodiments, or where inclusion of such structure/components is not necessary to an understanding of the various exemplary embodiments described herein. The lack of illustration/description of such structure/components in a particular figure is, however, not to be interpreted as limiting the scope of the various embodiments in any way.

### DETAILED DESCRIPTION

### Exemplary Microfluidic Devices

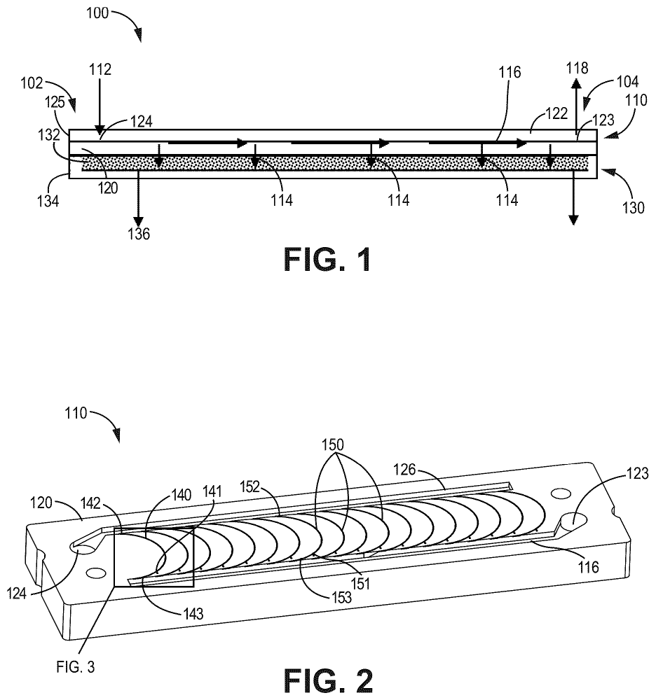

Some embodiments of the technology disclosed herein relate to a separation assembly 100, a schematic representation of which is depicted in FIG. 1. The separation assembly 100 generally has a microfluidic separator element 110 and a permeate draining layer 130 abutting the microfluidic separator element 110.

The microfluidic separator element 110 is generally configured to receive a liquid flow having dispersed particles and separate the liquid flow into two liquid streams. The microfluidic separator element 110 generally has a substrate layer 120, an element inlet 112, a plurality of first element outlets 114, and a second element outlet 118. The element inlet 112 is in direct fluid communication with the first element outlets 114 and the second element outlet 118. The element inlet 112 is generally configured to receive a flow a fluid that is to be separated, that is, a “feed” liquid stream. Each first element outlet 114 defines a fluid flow path for a first liquid stream of the separated fluid out of the separator element 110, such as a “permeate” liquid stream. The second element outlet 118 defines a fluid flow path for a second liquid stream of the separated fluid out of the separator element, such as a “retentate” liquid stream.

As used herein, the term “particle” refers to a discrete amount of material, which is dispersed in a fluid. Non-limiting examples of material that may be formed particles include dirt, metal, cells, air bubbles, fat, water droplets. In one particular example, water droplets may be dispersed in a hydrocarbon fluid, such as gasoline or diesel fuel, to form an emulsion. In another example, air bubbles may be dispersed in a hydraulic fluid. In another example, cells may be dispersed in an aqueous fluid. The cells can include eukaryotic cells. Example eukaryotic cells include mammalian cells, insect cells, plant cells, fungi cells, bacteria cells, and the like. Additional example eukaryotic cells include fish cells, crustacean cells, mollusk cells, and avian cells. In yet other examples, particles may be pulp in orange juice, fat in milk, and impurities in beer or wine.

The microfluidic separator element 110 is configured to concentrate suspended particles in the feed liquid stream such that each element outlet 114, 116 can accommodate one of, for example, a liquid stream having a relatively high particle concentration (a “particle laden” liquid stream) and a relatively low particle concentration (a “particle deficient” liquid stream). The targeted particles can be within a particular size range or can have a minimum size. For example, particles having a cross-dimension (such as diameter) ranging from 5-30 μm or 6-25 μm or 10-20 μm, can be particularly targeted by the microfluidic separator element 110, as examples. For non-spherical particles, for purposes of calculations provided herein, the particle diameter is considered to be the diameter of a volume-equivalent sphere. In some other examples, particles having a cross-dimension of at least 5 μm, at least 10 μm, at least 15 μm or at least 20 μm can be targeted by the microfluidic separator element 110. In some embodiments the first element outlet 114 is configured to receive a particle laden liquid stream and the second element outlet 118 is configured to receive a particle deficient liquid stream. In some other embodiments the second element outlet 118 is configured to receive a particle laden liquid stream and the first element outlet 114 is configured to receive a particle deficient liquid stream.

The microfluidic separator element 110 is defined as any type of microfluidic device that is configured to separate fluid having suspended particles into a particle laden liquid stream and a particle deficient liquid stream by liquid flowing through one or more flow channels defined by the microfluidic device. Each flow channel has a configuration that facilitates separation of a dispersed particle-containing liquid flowing therethrough into a particle laden liquid stream and the particle deficient liquid stream. In some embodiments the microfluidic separator element 110 has a plurality of flow channels. In some other embodiments the microfluidic separator element 110 has a single flow channel. In various embodiments, each flow channel has a height and a width that ranges from 20 to 1000 μm. In some embodiments, each of the flow channels are microchannels. In some embodiments the microfluidic separator element 110 is a hydrodynamic separator element, which is defined as a microfluidic device configured to focus particles within a liquid stream relying only on the forces due to internal liquid flow.

The microfluidic separator element 110 consistent with the technology disclosed herein generally has a substrate layer 120. The substrate layer 120 defines the flow channels therein. The substrate layer 120 can be constructed of a variety of different materials and combinations of materials. In various embodiments the substrate layer 120 is constructed of a non-permeable material, meaning that the substrate layer 120 does not accommodate liquid flow therethrough except through openings/pathways defined through the substrate layer 120 such as flow channels, inlets and outlets. The substrate layer 120 can be polymeric, in some embodiments. In some examples the substrate layer 120 is polydimethylsiloxane (PDMS). The substrate layer 120 can be constructed of polymers such as acrylic, polypropylene, polycarbonate, polyethylene, cyclic olefin copolymer (COC), and combinations thereof. In some embodiments the substrate layer 120 can include glass. In some embodiments the substrate layer 120 can include a non-reactive metal. In some embodiments the substrate layer 120 can include one or more adhesive layers, such as a pressure-sensitive adhesive. In some embodiments the substrate layer 120 is constructed of two or more materials.

It should be noted that in some embodiments, portions of the substrate layer 120 defining pathways to accommodate liquid flow, such as the flow channels, can have a material coating to limit particle adhesion to such pathways. Such coatings can include polyethylene glycol (PEG) chains, or nonionic surfactants, such as those available under the trade name PLURONIC from BASF Corporation. Such coatings can be particularly configured to limit cell adhesion through liquid flow pathways.

In some embodiments, the element inlet 112 of the microfluidic separator element 110 is defined towards a first lateral end 102 of the substrate layer 120 and the second element outlet 118 is defined towards a second lateral end 104 of the substrate layer 120. In such embodiments the microfluidic separator element 110 defines an outlet flow path 116 that extends laterally between the element inlet 112 and the second element outlet 118. In some embodiments the microfluidic separator element 110 defines a single first element outlet 114, but in other embodiments consistent with FIG. 1, the microfluidic separator element 110 defines a plurality of first element outlets 114. Each first element outlet 114 can be positioned laterally between the element inlet 112 and the second element outlet 118. The outlet flow path 116 generally extends laterally between the element inlet 112 and each of the first element outlet(s) 114. Flow channel configurations will be described in more detail below. In the current example, the second element outlet 118 facilitates liquid flow out of the assembly 100, and the first element outlet 114 facilitates liquid flow to the permeate draining layer 130. In various embodiments, each first element outlet 114 is an opening defined in the axial direction through the substrate layer 120 to the permeate draining layer 130.

### Permeate Draining Layer

The permeate draining layer 130 is configured to receive fluid from the first element outlets 114. As such, the permeate draining layer 130 is downstream of the first element outlets 114. The permeate draining layer 130 is in direct fluid communication with the first element outlets 114. The permeate draining layer 130 generally abuts the substrate layer 120. The permeate draining layer 130 generally extends laterally across the substrate layer 120. In some embodiments, the permeate draining layer 130 is coextensive to the substrate layer 120.

The permeate draining layer 130 defines a fluid volume 132 that is configured to receive a liquid. In some embodiments the fluid volume 132 is defined between the substrate layer 120 and a housing 134 of the permeate draining layer 130. In some other embodiments, the housing 134 of the permeate draining layer 130 encases the fluid volume 132, and the housing 134 defines one or more openings facilitating direct fluid communication between each first element outlet 114 and the fluid volume 132. In some embodiments the fluid volume 132 is a void volume, meaning an empty space. In some such embodiments, the housing 134 is a liquid impermeable layer of material and the fluid volume 132 is a fluid flow channel defined by the layer of material between the housing 134 and the substrate layer 120. The fluid flow channel extends laterally across the layer of material and extends across each of the first element outlets 114. The fluid flow channel is downstream of the plurality of first element outlets 114. In various embodiments, the fluid flow channel forming the fluid volume 132 is a microchannel. The fluid flow channel can have a length that is greater than or equal to the length across each of the first element outlets 114, in various embodiments.

In some other embodiments the fluid volume 132 is a porous material that is configured to receive a liquid. In some embodiments, one or more spacers can be disposed in the fluid volume between the substrate layer 120 and the housing 134 to maintain the size and shape of the fluid volume 132. In some embodiment the fluid volume 132 is defined by a mesh material disposed in the housing 134 of the permeate draining layer 130, where the mesh material defines a volume that is configured to receive a liquid.

The permeate draining layer 130 generally has a permeate layer outlet 136 that is configured to facilitate liquid flow out of the assembly 100. In some embodiments, the permeate draining layer 130 has a plurality of permeate layer outlets 136 that are each configured to facilitate liquid flow from the fluid volume 132 out of the assembly 100.

In some example implementations two or more separator assemblies consistent with FIG. 1 and the corresponding description can be arranged in parallel such that, for example, an element inlet 112 of another separation assembly that is generally consistent with the above-described separation assembly 100 is in fluid communication with the element inlet 112 of other separation assemblies within the group. In some such example implementations, the separator assemblies can be arranged in a stack, where the permeate draining layer 130 abuts a subsequent microfluidic separator element 110. The element inlet 112 of the subsequent microfluidic separator element 110 receives liquid flow in parallel with the element inlets of one or more other separator elements within the stack. In such a configuration, the particle concentration (whether particle laden or particle deficient) can be independently refined through each separation assembly 100.

In some alternative implementations, one or more separator elements can be arranged in series such that the particle concentration (whether particle laden or particle deficient) from one of the first element outlet 114 or the second element outlet 118 can be iteratively refined through each subsequent separation assembly 100. In some such example implementations, the separator assemblies can be arranged in a stack, where the permeate draining layer 130 abuts a subsequent microfluidic separator element 110, where the element inlet 112 of the subsequent microfluidic separator element 110 receives liquid flow from the permeate layer outlet 136 or the second element outlet 118. In such a configuration, the particle concentration (whether particle laden or particle deficient) from the relevant outlet 136/118 can be iteratively refined through each subsequent separation assembly 100. In some such examples, the permeate layer outlet 136 is in fluid communication with an element inlet 112 of another separation assembly. In some such alternate examples, the second element outlet 118 can be in fluid communication with an element inlet 112 of another separation assembly that is generally consistent with the above-described separation assembly 100.

It is noted that, while the second element outlet 118 depicted in FIG. 1 extends in the axial direction away from the permeate draining layer 130, the second element outlet 118 can extend in any direction, and in at least one example, the second element outlet 118 extends in the axial direction past the permeate draining layer 130 while bypassing the permeate draining layer 130.

### Exemplary Microfluidic Separator Element

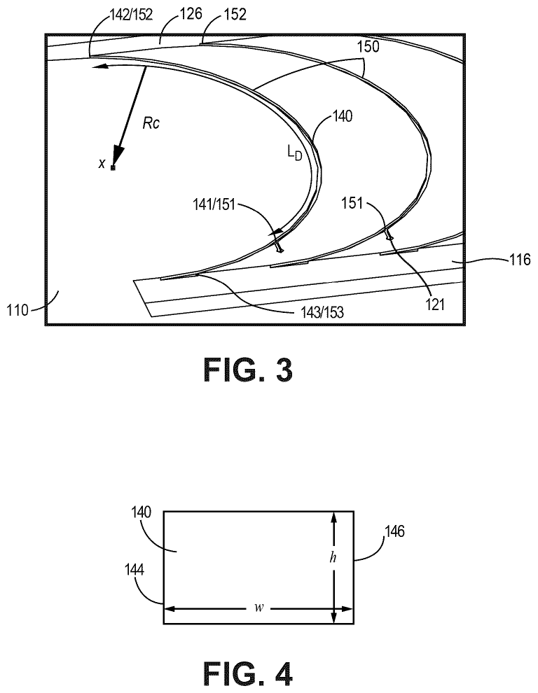

FIG. 2 is a perspective view of a portion of an exemplary substrate layer 120 of an example microfluidic separator element 110 consistent with FIG. 1, and FIG. 3 is a detail view of FIG. 2. More particularly, FIG. 2 is a perspective view of the substrate layer 120, which generally defines the flow channel(s) of the microfluidic separator element 110. In various examples, the microfluidic separator element 110 has a cover layer 122 (FIG. 1) sealed to the substrate layer 120 that seals an axial end of the flow channel(s) defined by the substrate layer 120. With particular reference to FIG. 2, the cover layer 122 of FIG. 1 would be configured to seal off the top side (relative to FIG. 2) of the flow channel(s). The cover layer 122 is omitted from FIG. 2 for visibility of the flow channel(s).

The microfluidic separator element 110 has a first microfluidic channel 140 defining a channel inlet 142 (FIG. 3) downstream of the element inlet 112 (FIG. 1) and a channel outlet including a first channel outlet 141 and a second channel outlet 143. The first microfluidic channel 140 is generally configured to accommodate liquid flow. The first channel outlet 141 is upstream of the first element outlet 114 (FIG. 1) and the second channel outlet 143 is upstream of the second element outlet 118 (FIG. 1). As discussed above, particles suspended in a liquid that are within a particular size range may be concentrated into one of the two channel outlets 141, 143 as a result of the liquid flowing from the channel inlet towards the channel outlets 141, 143.

The microfluidic channel 140 can be formed in the substrate layer 120 through molding operations, photolithography, and 3D printing, as examples. In some examples, the microfluidic channel 140 is formed in the substrate layer 120 through injection molding or embossing of plastics. Other approaches can also be used to form the microfluidic channel 140.

The first microfluidic channel 140 defines a channel length LD from the channel inlet 142 to the first channel outlet 141. The first microfluidic channel 140 has a length that is generally curved and defines an arc. Generally, the length of the first microfluidic channel 140 defines an arc of less than or equal to 270° and, in some embodiments, less than or equal to 200°. In various examples, such as those consistent with FIG. 3, the length of the first microfluidic channel 140 defines an arc of about 180° about a central axis x. The length of the first microfluidic channel 140 can be curved to define an inner radius R_{C} about the central axis x. As such, the length of the first microfluidic channel 140 can extend circumferentially about the central axis x. In the current example, the inner radius R_{C} is substantially constant along the length of the first microfluidic channel 140, but in some other examples, the inner radius R_{C} can vary. In the current example, the length of the first microfluidic channel 140 extends about 180° about the central axis x.

In the current example, the first microfluidic channel 140 defines a single curve, but in some other examples, the microfluidic channel 140 defines two or more curves that are connected. For example, the first microfluidic channel 140 can define two curves in opposite directions. One example of a first microfluidic channel 140 defining two curves in opposite directions would be an “s-shape” where a first portion of the first microfluidic channel 140 is curved in a first direction and a second portion of the first microfluidic channel 140 is curved in a second, opposite direction. Examples of such configurations will be described in more detail below.

The first microfluidic channel 140 generally has a rectangular cross-section along the channel length in the current example, which is visible in FIG. 4. The cross-section of the first microfluidic channel 140 is generally perpendicular to the direction of liquid flow through the first microfluidic channel 140. The first microfluidic channel 140 has a height (h) and a width (w) that is visible in FIG. 4. The first microfluidic channel 140 has an inner wall 144 and an outer wall 146, where the inner wall 144 is the wall defining the inner radius of curvature R_{C} and the outer wall 146 defines an outer radius. The width w of the first microfluidic channel 140 is the distance between the inner wall 144 and the outer wall 146. The first microfluidic channel 140 also has a hydraulic diameter (D_{H}). The following equation is used to calculate the hydraulic diameter of a microfluidic channel having a rectangular cross-section:

$${D}_{H} = \frac{2 \times height \times width}{height + width}$$

The first microfluidic channel 140 is configured to receive a liquid having a Reynolds number (Re) within the liquid channel. The liquid flow within a curving channel is described by two non-dimensional numbers, the Reynolds number and the Dean Number. The Reynolds number describes the ratio of inertial forces to viscous forces, and is defined as:

$$Re = \frac{ρ {UD}_{H}}{μ}$$

where ρ is the fluid density, U is the average fluid velocity, and μ is the dynamic viscosity of the fluid. In hydrodynamic separators the Reynolds number is typically small (<1000), which means that the flow profile is laminar. In various embodiments, the system is configured to have a Dean Number (De) between 5 and 25. In various embodiments, the system is configured to have a Dean Number between 5 and 20. The Dean number describes fluid behavior in a curved pipe and accounts for inertial forces, centripetal forces, and viscous forces acting on the fluid. The Dean number is defined as:

$$De = Re \sqrt{\frac{{D}_{H}}{2 {R}_{c}}}$$

The microfluidic separator element 110 is generally configured to concentrate particles in the first microfluidic channel 140. In various implementations, the separator element 110 is configured to concentrate particles having a diameter of greater than 8% of the hydraulic diameter of the first microfluidic channel 140. Particles whose diameter are greater than 8% of the channel hydraulic diameter are generally concentrated towards the inner wall when the Dean number ranges from 5 to 25. The hydrodynamic separator is generally configured to concentrate particles having a diameter that is less than or equal to 50% of the channel height. In various embodiments, hydrodynamic separators consistent with the technology disclosed herein are configured to concentrate particles having a density up to three times as dense as the liquid in the first microfluidic channel 140.

### Example Microfluidic Channels

In various embodiments consistent with the technology disclosed herein, the microfluidic separator element 110 has a plurality of microfluidic channels 150 including the first microfluidic channel 140. Each microfluidic channel of the plurality of microfluidic channels 150 is consistent with the description of the first microfluidic channel 140 herein. As such, each microfluidic channel of the plurality of microfluidic channels 150 defines a channel inlet 152 downstream of the element inlet 112 (FIG. 1), a first channel outlet 151 upstream of the first element outlet 114, and a second channel outlet 153 upstream of the second element outlet 118. Similarly, in various embodiments, each microfluidic channel of the plurality of microfluidic channels 150 has an inner wall and an outer wall and each microfluidic channel is configured to concentrate particles in a liquid stream towards the inner wall. The plurality of microfluidic channels 150 are generally arranged to operate in parallel with respect to liquid flow through the microfluidic separator element 110.

The plurality of microfluidic channels 150 are positioned relatively closely together to provide a relative increase in the liquid flow capacity that can be accommodated by the microfluidic separator element 110. In various embodiments the substrate layer 120 defines at least 4, at least 6, or at least 10 microfluidic channels.

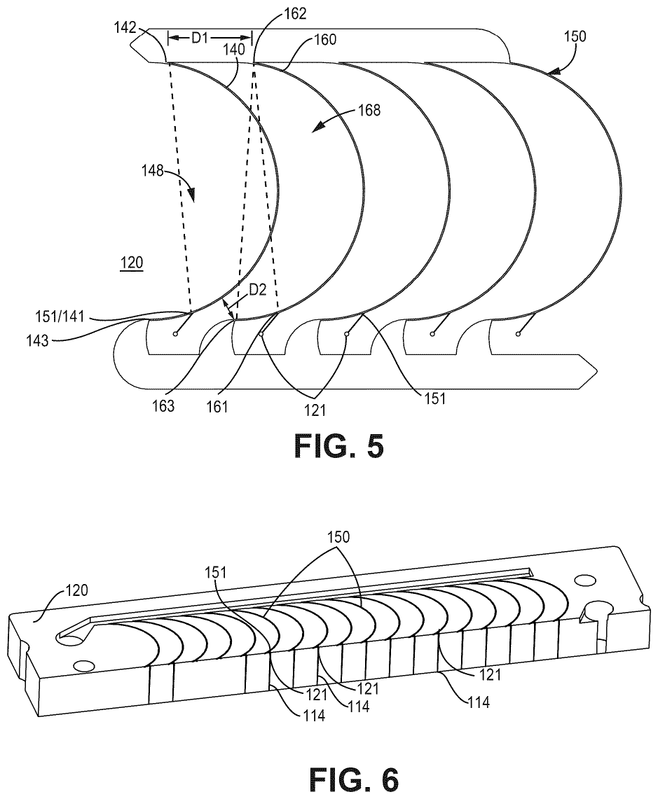

In various embodiments, the plurality of microfluidic channels 150 are arranged in a pattern in the lateral direction across the substrate layer 120. In some embodiments, the pattern is a regular pattern. In some embodiments, including that depicted in FIG. 2, each of the plurality of microfluidic channels 150 is nested with an adjacent microfluidic channel, meaning that each of the plurality of microfluidic channels 150 defines at least one arc along its length defining a concave region between the channel inlet 152 and a channel outlet 151, 153 that overlaps with a concave region of an adjacent channel. An example of this is visible in FIG. 5, which is a laterally facing view of a portion of the plurality of microfluidic channels 150 in a substrate layer 120 consistent with FIGS. 2 and 3. The first microfluidic channel 140 has a concave region 148 defined between the inlet 142 and the first channel outlet 141 (and second channel outlet 143) that is received by a concave region 168 that is defined by a second microfluidic channel 160 between its channel inlet 162 and first channel outlet 161, or by the 16 concave region defined between its channel inlet 162 and second channel outlet 163. Each of the plurality of microfluidic channels 150 is nested with an adjacent microfluidic channel.

In various embodiments each microfluidic channel 150 is offset in the lateral direction from adjacent microfluidic channels 150 across the substrate layer 120. The offset may define a particular offset distance D1 between corresponding locations along adjacent microfluidic channel 150. For example, the channel inlet 142 of the first microfluidic channel 140 can be spaced from the channel inlet 162 of the second microfluidic channel 160 by offset distance D1. Offset distance D1 is not particularly limited but will generally be less than the radius of curvature of a curve defined by a microfluidic channel. In some embodiments, D1 can be 20 mm or less, 18 mm or less, 16 mm or less, 14 mm or less, or even 12 mm or less. In some embodiments, D1 is constant, meaning that the lateral offset between adjacent microfluid channels 150 is substantially equal.

Each microfluidic channel of the plurality of microfluidic channels 150 can be positioned within a particular lateral distance D2 from an adjacent microfluidic channel, meaning that a first location along the length of a particular microfluidic channel 150 is within the particular lateral distance D2 of a second location along the length of an adjacent microfluidic channel, where the second location does not necessarily correspond to the first location As visible in FIG. 5, as an example, the lateral distance D2 between the second channel outlet 163 and the first microfluidic channel 140 is less than the offset distance D1 between the first microfluidic channel 140 and the second microfluidic channel 160. In some embodiments, the lateral distance D2 of a microfluidic channel is within 12 mm or within 10 mm of an adjacent microfluidic channel. Each microfluidic channel of the plurality of microfluidic channels 150 can be positioned within 5 mm of an adjacent microfluidic channel. Each microfluidic channel of the plurality of microfluidic channels 150 can be positioned within 3 mm of an adjacent microfluidic channel. Each microfluidic channel of the plurality of microfluidic channels 150 can be positioned within 2 mm of an adjacent microfluidic channel.

Returning again to FIGS. 2 and 3, the substrate layer 120 defines a layer inlet 124 (FIG. 2), a plurality of first layer outlets 121 (best visible in FIG. 3) and a second layer outlet 123 (FIG. 2). The layer inlet 124 is generally configured to receive liquid flow from outside of the substrate layer 120. The layer inlet 124 is upstream of each of the channel inlets 152. An inlet flow path 126 fluidly couples the layer inlet 124 to each of the channel inlets 152 including the channel inlet 142 of the first microfluidic channel 140. Each of the channel inlets 152 are disposed consecutively along the length of the inlet flow path 126. As such, fluid is configured to flow from the element inlet 112 (FIG. 1) through the layer inlet 124, along the inlet flow path 126, to the channel inlets 152. It is noted that, in some embodiments, the cross-sectional flow area defined by inlet flow path 126 can taper from the channel inlet 142 of the first microfluidic channel 140 to the channel inlet of the last microfluidic channel fluidly coupled to the inlet flow path 126. The taper of the cross-sectional flow area of the inlet flow path 126 can be configured to maintain a relatively constant average liquid flow velocity along the length of the inlet flow path 126, despite incremental liquid volume loss along the length of the inlet flow path 126 to each of the microfluidic channels 150. In some other embodiments the inlet flow path 126 has a cross-sectional flow area that remains constant along its length.

In examples consistent with FIG. 1, the layer inlet 124 of the substrate layer 120 receives liquid flow from the element inlet 112, where the element inlet 112 is defined by the cover layer 122. In some other embodiments the layer inlet is the element inlet, such as in embodiments where a liquid flow path extends laterally through an axial surface 125 of the substrate layer 120 to the inlet flow path 126.

Each first layer outlet 121 defines a fluid flow path out of the substrate layer 120. Each first channel outlet 151 is upstream of a first layer outlet 121. Each of the first channel outlets 151, including the first channel outlet 141 of the first microfluidic channel 140, is upstream of the first element outlet 114, which is shown schematically in FIG. 1 and a specific example of which is shown in more detail in FIG. 6, which is a cross-sectional view of the substrate layer 120 through each of the first channel outlets 151. In the current example, each first layer outlet 121 extends axially through the substrate layer 120 and fluidly couples a corresponding first channel outlet 151 to outside of the substrate layer 120. The first layer outlet 121 can be the same fluid flow path as the first element outlet 114. In various embodiments, such as those consistent with the schematic in FIG. 1, each first element outlet 114 fluidly couples a corresponding first channel outlet 151 to the permeate draining layer 130.

The second layer outlet 123 defines a fluid flow path out of the substrate layer 120. The microfluidic separator element 110 has an outlet flow path 116 (FIGS. 2 and 3) that is in fluid communication with the element inlet 112 and the second element outlet 118, where the example element inlet 112 and the example second element outlet 118 are depicted schematically in FIG. 1. More specifically, the outlet flow path 116 extends between the element inlet 112 and the second element outlet 118. The outlet flow path 116 is downstream of the element inlet 112. The outlet flow path 116 is downstream of each of the second channel outlets 153. The outlet flow path 116 is upstream of the second element outlet 118. As such, a portion of the liquid flowing through each of the plurality of microfluidic channels 150, such as a particle laden or a particle deficient liquid stream, exits through a corresponding second channel outlet 153 along the outlet flow path 116 through the second layer outlet 123 and out of the microfluidic separator element 110 through the second element outlet 118.

While the examples depicted in FIGS. 2, 3, 5 and 6 include a plurality of microfluidic channels 150 that operate in parallel, in some embodiments the microfluidic separator element 110 has a single microfluidic channel. In some embodiments microfluidic separator element 110 has one or more microfluidic channels having alternate shapes, such as having a lateral profile that is different than the arc depicted in the figures described earlier.

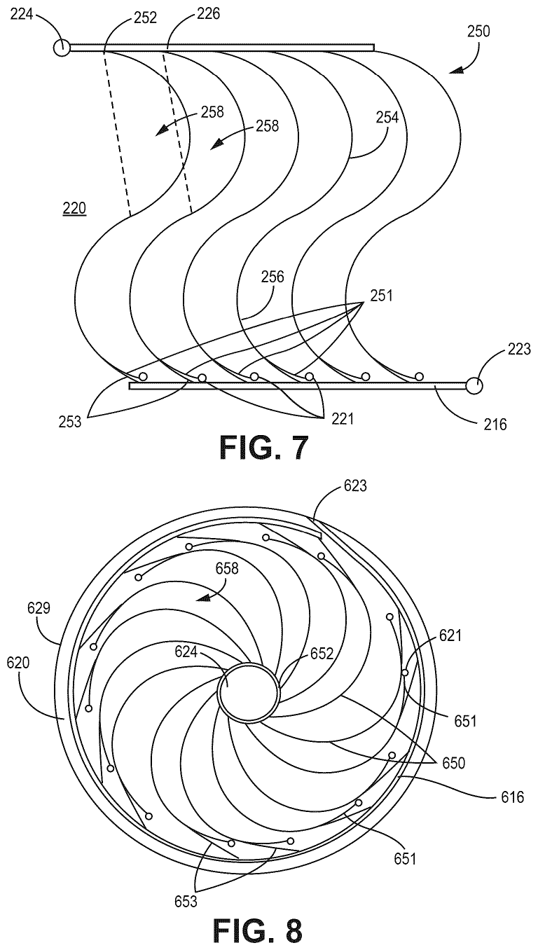

FIG. 7 is a laterally facing view of an example substrate layer 220 of a microfluidic separator element 110 (such as the example of FIG. 1) having a plurality of microfluidic channels 250 consistent with the technology disclosed herein. Each microfluidic channel of the plurality of microfluidic channels 250 is generally consistent with the descriptions above of microfluidic separator elements and microfluidic channels, which are incorporated by reference here, except where contradictory to the current description or example depicted in FIG. 7.

A hydrodynamic separator element has a substrate layer 220 defining a layer inlet 224, a plurality of first layer outlets 221, and a second layer outlet 223. The substrate layer 220 is generally non-permeable. The substrate layer 220 defines an outlet flow path 216 upstream of the second layer outlet 223. The substrate layer 220 defines a plurality of curved microfluidic channels 250 arranged to operate in parallel. In various embodiments the present technology includes at least 6 microfluidic channels 250 in the substrate layer 220. Each microfluidic channel 250 defines a channel inlet 252 downstream of the layer inlet 224. An inlet flow path 226 fluidly couples the layer inlet 224 and the channel inlet 252 in the current example, which can be consistent with descriptions of inlet flow paths elsewhere herein. Each microfluidic channel 250 defines a first channel outlet 251 upstream of a first layer outlet 221 of the plurality of first layer outlets. Each microfluidic channel 250 has a second channel outlet 253 that is upstream of the outlet flow path 216.

When a separator element having a substrate layer consistent with the present example is incorporated in an assembly consistent with FIG. 1, it is noted that each channel inlet 252 is configured to be downstream of the element inlet 112 (FIG. 1), the first channel outlet 251 is configured to be upstream of the first element outlet 114 (FIG. 1), and the second channel outlet 253 is configured to be upstream of the second element outlet 118 (FIG. 1). The plurality of microfluidic channels 250 are generally arranged to operate in parallel with respect to liquid flow through the microfluidic separator element. As previously described, each microfluidic channel of the plurality of microfluidic channels 250 has an inner wall and an outer wall (consistent with FIG. 4) and each microfluidic channel 250 can be configured to concentrate particles in a liquid stream towards the inner wall. The particles can range from 10-20 microns. In some examples, the particles are eukaryotic cells such as mammalian or insect cells or others as has been described above.

Similar to examples discussed above, each of the plurality of microfluidic channels 250 are curved. In particular, the length of each microfluidic channel is curved between the channel inlet 252 and the channel outlets 251, 253. In the current example, each microfluidic channel 250 defines two curves in opposite directions, meaning that each microfluidic channel 250 has a first length 254 that is outwardly curved (or curved towards a first lateral direction) and a second length 256 that is inwardly curved (or curved towards a second lateral direction opposite the first lateral direction). Other curved shapes are also possible.

Similar to examples discussed above, in the current example, the length of each of the microfluidic channels defines an arc of less than or equal to 270° or less than or equal to 200°. As is visible, in the current design each of the plurality of microfluidic channels 250 is nested with an adjacent microfluidic channel, which may advantageously maximize the volume of liquid flow that can be accommodated by the substrate layer. In particular, as visible in FIG. 7, each microfluidic channel 250 defines at least one arc along its length defining a concave region 258 between the channel inlet 252 and a channel outlet 251, 253 that overlaps with a concave region 258 of an adjacent microfluidic channel. In the current example, each microfluidic channel is within 12 mm, 10 mm, 5 mm, 3 mm, or even 2 mm of an adjacent microfluidic channel. In some embodiments, each microfluidic channel is within 12 mm or within 10 mm of an adjacent microfluidic channel along at least 50% of the length of the microfluidic channel. In some embodiments, each microfluidic channel is within 5 mm of an adjacent microfluidic channel along at least 50% of the length of the microfluidic channel. In some embodiments, each microfluidic channel is within 3 mm of an adjacent microfluidic channel along at least 50% of the length of the microfluidic channel. In some embodiments, each microfluidic channel is within 2 mm of an adjacent microfluidic channel along at least 50% of the length of the microfluidic channel.

FIG. 8 is a laterally facing view of yet another example substrate layer 620 of a microfluidic separator element 110 (such as the example of FIG. 1) having a plurality of microfluidic channels 650 consistent with the technology disclosed herein. Each microfluidic channel of the plurality of microfluidic channels 650 is generally consistent with the descriptions above of microfluidic separator elements and microfluidic channels, which are incorporated by reference here, except where contradictory to the current description or the example depicted in FIG. 8.

A hydrodynamic separator element has a substrate layer 620 defining a layer inlet 624, a plurality of first layer outlets 621, and a second layer outlet 623. The substrate layer 620 is generally non-permeable. In the current example, the substrate layer 620 has a lateral profile that is circular in shape, although the substrate layer 620 can have alternate lateral shapes. Unlike examples described above, in the current example, the layer inlet 624 is directly fluidly coupled to each of the channel inlets 652, and thus there is no separate inlet flow path. In the current example, the layer inlet 624 is defined by an opening in the substrate layer 620 that is central to all of the channel inlets 652 of the plurality of microfluidic channels 650. In some other embodiments the layer inlet 624 can be a discrete opening and the substrate can define an inlet flow path that fluidly couples the layer inlet 624 to each channel inlet 652 in sequence.

The substrate layer 620 defines an outlet flow path 616 upstream of the second layer outlet 623. In the current example, the lateral profile of the outlet flow path 616 is circular in shape. The length of the outlet flow path 616 is positioned in the radial direction between the plurality of microfluidic channels 650 and an outer perimetric boundary 629 of the substrate layer 620. The substrate layer 620 defines a plurality of curved microfluidic channels 650 arranged to operate in parallel. In various embodiments the present technology includes at least 6 or at least 10 microfluidic channels 650 in the substrate layer 620. Each microfluidic channel 650 defines a channel inlet 652 downstream of the layer inlet 624. Each microfluidic channel 650 defines a first channel outlet 651 upstream of a first layer outlet 621 of the plurality of first layer outlets. Each microfluidic channel 650 has a second channel outlet 653 that is upstream of the outlet flow path 616. In some alternate embodiments, an outlet flow path 616 can be omitted where the layer outlet is the outer perimetric boundary 629 of the substrate 620 and the second channel outlet 653 extends to the outer perimetric boundary 629.

As discussed above with respect to FIG. 7, the channel inlet 652 is configured to be downstream of the element inlet 112 (FIG. 1), the first channel outlet 651 is configured to be upstream of the first element outlet 114 (FIG. 1), and the second channel outlet 653 is configured to be upstream of the second element outlet 118 (FIG. 1). The plurality of microfluidic channels 650 are generally arranged to operate in parallel with respect to liquid flow through the microfluidic separator element. As previously described, each microfluidic channel of the plurality of microfluidic channels 650 has an inner wall and an outer wall (consistent with FIG. 4) and each microfluidic channel 650 can be configured to concentrate particles in a liquid stream towards the inner wall. The particles can range from 10-20 microns. In some examples, the particles are eukaryotic cells such as mammalian or insect cells.

Similar to examples discussed above, each of the plurality of microfluidic channels 650 are curved. In particular, the length of each microfluidic channel is curved between the channel inlet 652 and the channel outlets 651, 653. In the current example, each microfluidic channel 650 defines a single curve, although more complex curves, such as that depicted in FIG. 7 can be employed. Unlike some other examples, each microfluidic channel 650 is curved towards a different lateral direction. In particular, each microfluidic channel 650 extends radially outward from the layer inlet 624 towards an outer perimetric boundary 629 of the substrate layer 620. The channel inlets 652 of the microfluidic channels 650 are spaced perimetrically around a central area of the substrate layer 620 (which in this example is the layer inlet 624). The channel outlets 651, 653 are spaced perimetrically around an outer area of the substrate layer 620. Each of the plurality of microfluidic channels 650 can define an identical curve, and each arc is angularly offset from adjacent microfluidic channels 650.

Similar to examples discussed above, in the current example, the length of each of the microfluidic channels defines an arc of less than or equal to 270° or less than or equal to 200°. As is visible, in the current design each of the plurality of microfluidic channels 650 is nested with an adjacent microfluidic channel, which may advantageously maximize the volume of liquid flow that can be accommodated by the substrate layer. In particular, as visible in FIG. 8, each microfluidic channel 650 defines at least one arc along its length defining a concave region 658 between the channel inlet 652 and a channel outlet 651, 653 that overlaps with a concave region 658 of an adjacent microfluidic channel. In the current example, each microfluidic channel is within 12 mm, 10 mm, 5 mm, or even 3 mm of an adjacent microfluidic channel. In some embodiments, each microfluidic channel is within 12 mm, 10 mm, 5 mm, or even 3 mm of an adjacent microfluidic channel along at least 50% of the length of the microfluidic channel.

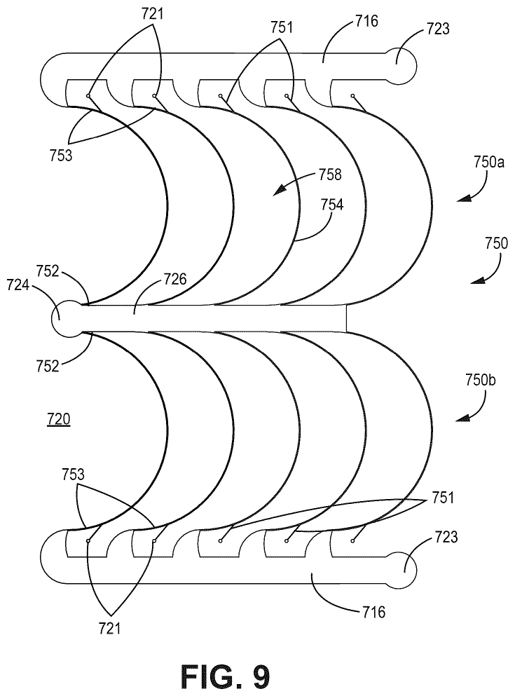

FIG. 9 is a laterally facing view of yet another example substrate layer 720 of a microfluidic separator element 110 (such as the example of FIG. 1) having a plurality of microfluidic channels 750 consistent with the technology disclosed herein. Each microfluidic channel of the plurality of microfluidic channels 750 is generally consistent with the descriptions above of microfluidic separator elements and microfluidic channels, which are incorporated by reference here, except where contradictory to the current description or example depicted in FIG. 9.

A hydrodynamic separator element has a substrate layer 720 defining a layer inlet 724, a plurality of first layer outlets 721, and a second layer outlet 723. Unlike previous examples, here the substrate layer 720 defines two second layer outlets 723. The substrate layer 720 defines an outlet flow path 716 upstream of each second layer outlet 723. The substrate layer 720 defines a plurality of curved microfluidic channels 750 arranged to operate in parallel. In various embodiments the present technology includes at least 6 microfluidic channels 750 in the substrate layer 720. Each microfluidic channel 750 defines a channel inlet 752 downstream of the layer inlet 724. An inlet flow path 726 fluidly couples the layer inlet 724 and the channel inlet 752 in the current example. The inlet flow path 726 can have a cross-sectional flow area that is tapered along its length. Each microfluidic channel 750 defines a first channel outlet 751 upstream of a first layer outlet 721 of the plurality of first layer outlets. Each microfluidic channel 750 has a second channel outlet 753 that is upstream of the outlet flow path 716.

When a separator element having a substrate layer consistent with the present example is incorporated in an assembly consistent with FIG. 1, it is noted that each channel inlet 752 is configured to be downstream of the element inlet 112 (FIG. 1), the first channel outlet 751 is configured to be upstream of the first element outlet 114 (FIG. 1), and each second channel outlet 753 is configured to be upstream of the second element outlet 118 (FIG. 1). The plurality of microfluidic channels 750 are generally arranged to operate in parallel with respect to liquid flow through the microfluidic separator element. As previously described, each microfluidic channel of the plurality of microfluidic channels 750 has an inner wall and an outer wall (consistent with FIG. 4) and each microfluidic channel 750 can be configured to concentrate particles in a liquid stream towards the inner wall. The particles can range from 10-20 microns. In some examples, the particles are eukaryotic cells such as those examples described above.

Similar to examples discussed above, each of the plurality of microfluidic channels 750 are curved. In particular, the length of each microfluidic channel is curved between the channel inlet 752 and the channel outlets 751, 753. Each microfluidic channel 750 has a length 754 that is curved towards a first lateral direction. Other curved shapes are also possible, as has been described above.

In the current example, the plurality of microfluidic channels 750 defined by the substrate 720 includes a first set of microfluidic channels 750a and a second set of microfluidic channels 750b. The first set of microfluidic channels 750a have channel inlets 752 that are opposite the channel inlets 752 of the second set of microfluidic channels 750b relative to the inlet flow path 726. In the current example, the first set of microfluidic channels 750a are curved in the same lateral direction as the second set of microfluidic channels 750b. In some other embodiments, the first set of microfluidic channels 750a are curved in a different lateral direction than the second set of microfluidic channels 750b. For example, the first set of microfluidic channels 750a can be curved in the opposite lateral direction of the second set of microfluidic channels 750b. In this example, the first set of microfluidic channels 750a have a nested configuration and the second set of microfluidic channels 750b have a nested configuration. The first set of microfluidic channels 750a are not nested with the second set of microfluidic channels 750b, however.

Similar to examples discussed above, in the current example, the length of each of the microfluidic channels defines an arc of less than or equal to 270° or less than or equal to 200°. As is visible, in the current design each of the plurality of microfluidic channels 750 is nested with an adjacent microfluidic channel, which may advantageously maximize the volume of liquid flow that can be accommodated by the substrate layer. In particular, as visible in FIG. 7, each microfluidic channel 750 defines at least one arc along its length defining a concave region 758 between the channel inlet 752 and a channel outlet 751, 753 that overlaps with a concave region 758 of an adjacent microfluidic channel. In the current example, each microfluidic channel is within 12 mm, 10 mm, 5 mm, or even 3 mm of an adjacent microfluidic channel. In some embodiments, each microfluidic channel is within 12 mm, 10 mm, 5 mm, or even 3 mm of an adjacent microfluidic channel along at least 50% of the length of the microfluidic channel.

### Example Implementations

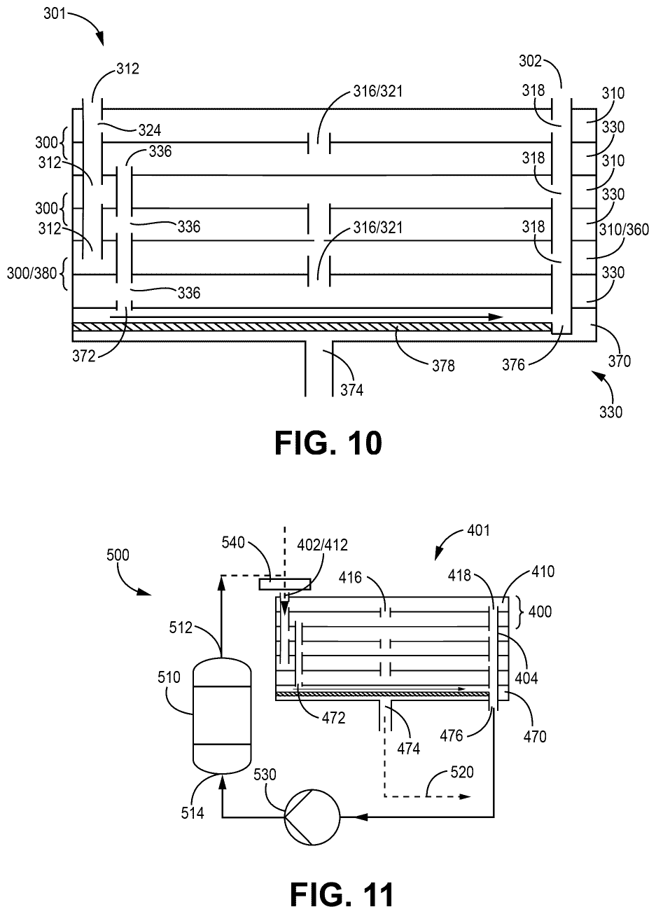

Example microfluidic separator elements and separation assemblies consistent with the technology disclosed herein may advantageously be incorporated in a variety of different systems. One example system 301 is depicted schematically in FIG. 10, which can incorporate one or more separator elements 310 consistent, for example, with FIG. 1. Each separator element 310 can incorporate a substrate layer consistent with substrate layers discussed elsewhere herein, particularly with references any of FIGS. 2-9. The corresponding descriptions above related to the separator elements and relevant components are incorporated herein by reference.

In the current example, the system 301 disclosed herein can be consistent with a filtration cassette in a variety of implementations. In the current example system, at least one separator element 310 is positioned in series with a tangential flow filter 370. The separator element 310 is positioned upstream of the tangential flow filter 370. Positioning the separator element 310 upstream and in series with the tangential flow filter 370 may advantageously extend the life of the tangential flow filter 370 by slowing down the fouling of filter media 378 within the tangential flow filter 370. In the current example, the separator element 310 functions as a pre-filter for the tangential flow filter 370.

The separator element 310 can be a hydrodynamic separator element in various embodiments. The separator element 310 has an element inlet 312, a plurality of first element outlets 316, a second element outlet 318. While not currently visible, the separator element 310 can have a plurality of curved microfluidic channels between the element inlet 312 and the first element outlets 316. Each element inlet 312 is in direct fluid communication with, and upstream of, a corresponding layer inlet 324. The curved microfluidic channels are consistent with the curved microfluidic channels discussed elsewhere herein. For example, each curved microfluidic channel can have an inner wall 144 and an outer wall 146 (FIG. 4). In various embodiments, the separator element 310 is configured to concentrate particles along the inner wall. Such embodiments may require lower pressure drop and liquid flow rate to separate particles compared to systems that concentrate particles on the outer wall, which may advantageously preserve cell viability where the separator element 310 is configured to concentrate particles such as cells.

The tangential flow filter 370 can be consistent with a variety of tangential flow filters known in the art. In general, the tangential flow filter 370 has a feed inlet 372, a retentate outlet 376, and a permeate outlet 374. The feed inlet 372 is downstream of the first element outlets 316. Filter media 378 is disposed between the feed inlet 372 and the permeate outlet 374. As is generally known in the art, liquid having suspended particles is flowed across the surface of the filter media 378. Permeate is configured to pass through the filter media 378 to the permeate outlet 374. Retentate is configured to flow along the surface of the filter media 378, rather than through the filter media 378, to the retentate outlet 376.

Consistently with examples discussed elsewhere herein, the separator element 310 can have a substrate layer defining a layer inlet, a plurality of first layer outlets and a second layer outlet. The layer inlet is downstream of the element inlet 312. Each first layer outlet is upstream of a first element outlet 316. The second layer outlet is upstream of the second element outlet 318. In some embodiments, each substrate layer can define a single microfluidic channel. In other embodiments, the substrate layer of each separator element 310 can define a plurality of curved microfluidic channels extending between the element inlet 312 and each element outlet 316, 318. The plurality of curved microfluidic channels can extend between a layer inlet and a first layer outlet of the plurality of first layer outlets of the substrate layer of the separator element 310, as discussed above. The plurality of curved microfluidic channels can be consistent with descriptions and figures elsewhere herein.

In various embodiments, the system has a plurality of separator elements 310. The separator elements 310 are in a stacked configuration with the tangential flow filter 370. As such, each of the substrate layers of the separator elements 310 are in a stacked configuration as well. In various embodiments, a plurality of separator elements 310 are in a stacked configuration with the tangential flow filter 370. The plurality of separator elements 310 are arranged in parallel in various embodiments. In some examples, such as those consistent with FIG. 10, each first element outlet 316 of each separator element 310 is upstream of, and fluidly coupled to, a permeate layer outlet 336. The permeate layer outlets 336 of each separator element 310 are in fluid communication. The permeate layer outlets 336 are each upstream of, and fluidly coupled to, the feed inlet 372 of the tangential flow filter 370. Collectively, the plurality of separator elements 310 are arranged in series with the tangential flow filter 370.

In some examples, each separator element 310 is a component of a separation assembly 300. Each separation assembly 300 can also have a permeate draining layer 330 that is downstream of a plurality of first layer outlets 321 and a plurality of first element outlets 316 of the separator element 310. The permeate draining layer 330 can abut the substrate layer as described elsewhere herein. The permeate draining layer 330 can extend laterally across a corresponding substrate layer of an abutting separator element 310. The permeate draining layer 330 is consistent with figures and descriptions elsewhere herein.

In various embodiments, each separation assembly 300 is arranged in parallel with other separator assemblies 300 in the system 301. In various embodiments, a plurality of separator assemblies 300 are in a stacked configuration with the tangential flow filter 370. The plurality of separator assemblies 300 are arranged in series with the tangential flow filter 370. In some examples, such as those consistent with FIG. 10, the permeate draining layer 330 has a permeate layer outlet 336 that is upstream of, and fluidly coupled to, feed inlet 372 of the tangential flow filter 370.

While the current example depicts three example separator assemblies 300 arranged in a stack, it will be appreciated that hundreds of separator assemblies 300 can be included in separator systems consistent with those disclosed herein. In some embodiments, a separator system disclosed herein has 10-30, 20-60, 50-100, or 100-200 separator assemblies 300 arranged in parallel. It will be appreciated that each separation assembly 300 can have a height ranging from 1 mm to 10 mm or 2 mm to 5 mm in some embodiments.

In the current example, each second element outlet 318 of each separator element 310 is in fluid communication with the second element outlet 318 of the other separator elements 310 in the system 301. In particular, an outlet flow line 302 is defined by each of the separator elements 310. Each second element outlet 318 is in direct fluid communication with the outlet flow line 302. Further, in the current example, the retentate outlet 376 of the tangential flow filter 370 is also in direct fluid communication with the outlet flow line 302.

In various embodiments, each separation assembly 300 in the system 301 has a lateral profile that is coextensive with the lateral profile of the tangential flow filter 370. In various embodiments, each separation assembly 300 has a configuration that accommodates inclusion in a tangential flow filter cassette assembly.

In some example implementations, the system 301 is configured to separate particles having diameters ranging from 10-20 microns from a liquid stream. In various example implementations, the system 301 is configured to separate eukaryotic cells in a liquid stream, such as those example eukaryotic cells described above. In some such example implementations, the system 301 is a component of a cell retention device of a perfusion bioreactor. The perfusion bioreactor may generally be used in the production of biotherapeutics. A schematic representation of such a perfusion bioreactor is depicted schematically in FIG. 11, which will now be described.

The perfusion bioreactor 500 generally has a cell culture tank 510, a microfluidic separator element 410, and a tangential flow filter 470. The microfluidic separator element 410 is a component of a separation assembly 400, which is a component of a separator system 401 such as that described above with reference to FIG. 10. The tangential flow filter 470 is a component of the separator system 401, also consistent with the description of FIG. 10. It will be appreciated that in some examples, the tangential flow filter 470 and the microfluidic separator element 410 can be discrete components, and in other example, the tangential flow filter 470 and the microfluidic separator element 410 can be integrated in a single component assembly, such as a cassette.

The cell culture tank 510 is generally configured to contain a liquid medium and cells distributed within the liquid medium. The cell culture tank is generally configured to facilitate the culturing of the cells. The perfusion bioreactor 500 is generally configured to circulate the liquid medium through the system to maintain favorable culturing conditions within the cell culture tank 510.

The separator system 401 generally has a system inlet 402 that is configured to receive a particle containing liquid stream, such as a liquid stream containing cells. The system inlet 402 is in direct fluid communication with each element inlet 412 of each separator element 400. In some embodiments, the system inlet 402 can be an element inlet 412 of a separator element 400, which has been described in detail above. In the current example, the system inlet 402 is the inlet of the upstream-most separator element 400 of the plurality of separator elements 400. The liquid stream can be received from the cell culture tank 510 in some implementations, and in other implementations the liquid stream can be received from another source. In various embodiments, a pre-filtration element 540 can be positioned upstream of the system inlet 402. The pre-filtration element 540 can be a component of the separator system 401 and can be coupled to the separator element 400 upstream of the system inlet 402. In some other embodiments the pre-filtration element 540 is a separate component from the separator element 400 and can be in fluid communication with a liquid flow path upstream of the system inlet 402. The pre-filtration element 540 can be particularly configured to capture relatively large particles such as cell agglomeration that may otherwise obstruct liquid pathways of the separator element 400.

The liquid stream can flow through each separator element 410 in parallel. As such, the element inlets 412 are arranged in parallel relative to the system inlet 402. Within each separator element 410 the liquid stream can flow through the plurality of curved microfluidic channels in parallel, as has been discussed above. Each of the microfluidic channels separate the liquid stream into a particle laden liquid stream and a particle deficient liquid stream. The particle deficient portion of the liquid stream can pass through each first channel outlet and each first element outlet 416 as discussed above. Such particle deficient portion of the liquid stream can exit each first element outlet 416 and enter the feed inlet 472 of the abutting permeate draining layer 330. The particle deficient portion of the liquid stream is configured to flow through the feed inlet 472 of the tangential flow filter 470, which further separates the particle deficient portion of the liquid stream into a particle laden and particle deficient liquid stream. The particle deficient portion of the liquid stream is configured to flow through the permeate outlet 474 of the tangential flow filter 470 and flows along a flow path 520 to an external system for processing or disposal.

The particle laden portion of the liquid stream, as an example, can exit each separator element 410 through its respective second element outlet 418. The particle laden liquid stream is configured to pass through the second outlet 418 of corresponding separator element 410 to an outlet flow line 404 which allows the liquid stream to exit the separator system 401. Similarly, the retentate fluid stream can exit the tangential flow filter 470 through a retentate outlet 476. The retentate outlet 476 can also be in fluid communication with the outlet flow line 404. In some such example implementations, the cell culture tank 510 is positioned downstream of, and in fluid communication with, the tangential flow filter 470. The cell culture tank 510 can also be positioned downstream of, and in fluid communication with, each of the separator elements 410. The cell culture tank 510 is positioned downstream of, and in fluid communication with, the separator system 401 generally. The cell culture tank 510 will generally have an inlet 514 that is in fluid communication with the outlet flow line 404 of the separator system 401.

As has been mentioned above, in some embodiments the cell culture tank 510 can also be positioned upstream of the separator system 401 in some embodiments. In such an embodiment the cell culture tank 510 would have an outlet 512 that is in fluid communication with the inlet 402 of the separation assembly 401.

In various implementations, a liquid pump 530 is fluidly coupled to the separator system 401 and the cell culture tank 510 to facilitate liquid flow through the system 500.

### Exemplary Aspects

Aspect 1. A separation assembly comprising: a microfluidic separation element comprising a substrate layer, wherein the microfluidic separation element defines: an element inlet, a plurality of first element outlets defined by the substrate layer, and a second element outlet; and a permeate draining layer abutting the substrate layer, wherein the permeate draining layer is downstream of the plurality of first element outlets.

Aspect 2. The separation assembly of any one of aspects 1 and 3-21, wherein the permeate draining layer extends laterally across the substrate layer.

Aspect 3. The separation assembly of any one of aspects 1-2 and 4-21, wherein the separation assembly is a component of a cell retention device of a perfusion bioreactor.

Aspect 4. The separation assembly of any one of aspects 1-3 and 5-21, wherein the microfluidic separation element is a hydrodynamic separator element.

Aspect 5. The separation assembly of any one of aspects 1-4 and 6-21, wherein the permeate draining layer is defined by a mesh material.

Aspect 6. The separation assembly of any one of aspects 1-5 and 7-21, wherein the permeate draining layer is a material layer defining a microchannel downstream of the plurality of first layer outlets.

Aspect 7. The separation assembly of any one of aspects 1-6 and 8-21, further comprising: a first microfluidic channel defining a channel inlet downstream of the element inlet and a channel outlet having a first channel outlet upstream of a first element outlet of the plurality of first channel outlets and a second channel outlet upstream of the second element outlet.

Aspect 8. The separation assembly of any one of aspects 1-7 and 9-21, wherein the first microfluidic channel is curved.

Aspect 9. The separation assembly of any one of aspects 1-8 and 10-21, further comprising a plurality of microfluidic channels including the first microfluidic channel, wherein each microfluidic channel defines a channel inlet downstream of the element inlet and a first channel outlet upstream of a first element outlet of the plurality of first element outlets and a second channel outlet upstream of the second element outlet.

Aspect 10. The separation assembly of any one of aspects 1-9 and 11-21, wherein each of the plurality of microfluidic channels is nested with an adjacent microfluidic channel.

Aspect 11. The separation assembly of any one of aspects 1-10 and 12-21, wherein the plurality of microfluidic channels comprises at least 6 microfluidic channels.

Aspect 12. The separation assembly of any one of aspects 1-11 and 13-21, wherein each microfluidic channel is within 12 mm, 10 mm, 5 mm, or 3 mm of an adjacent microfluidic channel.

Aspect 13. The separation assembly of any one of aspects 1-12 and 14-21, wherein each microfluidic channel is curved.

Aspect 14. The separation assembly of any one of aspects 1-13 and 15-21, wherein each microfluidic channel defines two curves in opposite directions.

Aspect 15. The separation assembly of any one of aspects 1-14 and 16-21, wherein each microfluidic channel has an inner wall and an outer wall and each microfluidic channel is configured to concentrate particles in a liquid stream towards the inner wall.

Aspect 16. The separation assembly of any one of aspects 1-15 and 17-21, wherein the length of each of the plurality of microfluidic channels defines an arc of less than or equal to 270°.

Aspect 17. The separation assembly of any one of aspects 1-16 and 18-21, wherein the length of each of the plurality of microfluidic channels defines an arc of less than or equal to 200°.

Aspect 18. The separation assembly of any one of aspects 1-17 and 19-21, wherein the microfluidic separation element is configured to separate particles ranging from 10-20 microns from a liquid stream.

Aspect 19. The separation assembly of any one of aspects 1-18 and 20-21, wherein the microfluidic separation element is configured to separate one or both of mammalian cells and insect cells in a liquid stream.

Aspect 20. The separation assembly of any one of aspects 1-19 and 21, wherein the microfluidic separation element is configured to separate one or both of fish cells and avian cells in a liquid stream.

Aspect 21. A system comprising: a plurality of separation assemblies of any one of aspects 1-20, wherein the separation assemblies are arranged in a stacked configuration; and a system inlet in direct fluid communication with each element inlet, wherein the plurality of separation assemblies are arranged to operate in parallel.

Aspect 22. A hydrodynamic separator element comprising: a substrate layer defining: a layer inlet, a plurality of first layer outlets and a second layer outlet, an outlet flow path upstream of the second layer outlet, and a plurality of curved microfluidic channels arranged to operate in parallel, wherein each microfluidic channel defines: a channel inlet downstream of the layer inlet, a first channel outlet upstream of a first layer outlet of the plurality of first layer outlets, and a second channel outlet upstream of the outlet flow path, wherein the substrate layer is non-permeable, and wherein each of the plurality of microfluidic channels is nested with an adjacent microfluidic channel.

Aspect 23. The hydrodynamic separator element of any one of aspects 22 and 24-38, wherein the length of each of the plurality of curved microfluidic channels defines an arc of less than or equal to 270°.

Aspect 24. The hydrodynamic separator element of any one of aspects 22-23 and 25-38, wherein the length of each of the plurality of curved microfluidic channels defines an arc of less than or equal to 200°.

Aspect 25. The hydrodynamic separator element of any one of aspects 22-24 and 26-38, wherein each microfluidic channel defines two curves in opposite directions.

Aspect 26. The hydrodynamic separator element of any one of aspects 22-25 and 27-38, wherein the plurality of curved microfluidic channels is at least 6 microfluidic channels.

Aspect 27. The hydrodynamic separator element of any one of aspects 22-26 and 28-38, wherein each microfluidic channel is within 12 mm, 10 mm, 5 mm, or 3 mm of an adjacent microfluidic channel.

Aspect 28. The hydrodynamic separator element of any one of aspects 22-27 and 29-38, further comprising a permeate draining layer abutting the substrate layer, wherein the permeate draining layer is downstream of the first layer outlets.

Aspect 29. The hydrodynamic separator element of any one of aspects 22-28 and 30-38, wherein the permeate draining layer extends laterally across the substrate layer.

Aspect 30. The hydrodynamic separator element of any one of aspects 22-29 and 31-38, wherein the permeate draining layer is defined by a mesh material.

Aspect 31. The hydrodynamic separator element of any one of aspects 22-30 and 32-38, wherein the permeate draining layer is a material layer defining a microchannel downstream of the plurality of first layer outlets.

Aspect 32. The hydrodynamic separator element of any one of aspects 22-31 and 33-38 wherein the hydrodynamic separator element is a component of a cell retention device of a perfusion bioreactor.

Aspect 33. The hydrodynamic separator element of any one of aspects 22-32 and 34-38, wherein each microfluidic channel has an inner wall and an outer wall and each microfluidic channel is configured to concentrate particles in a liquid stream towards the inner wall.

Aspect 34. The hydrodynamic separator element of any one of aspects 22-33 and 35-38, wherein the hydrodynamic separator element is configured to separate particles ranging from 10-20 microns from a liquid stream.

Aspect 35. The hydrodynamic separator element of any one of aspects 22-34 and 36-38, wherein the hydrodynamic separator element is configured to separate one or both of mammalian cells and insect cells in a liquid stream.

Aspect 36. The hydrodynamic separator element of any one of aspects 22-35 and 37-38, wherein the hydrodynamic separator element is configured to separate one or both of fish cells and avian cells in a liquid stream.

Aspect 37. The hydrodynamic separator element of any one of aspects 22-36 and 38, further comprising: a plurality of the substrate layers in a stacked configuration; and a system inlet in direct fluid communication with each layer inlet, wherein the plurality of substrate layers are arranged to operate in parallel.

Aspect 38. The hydrodynamic separator element of any one of aspects 22-37, further comprising a permeate draining layer abutting each substrate layer, wherein each permeate draining layer is downstream of a corresponding first layer outlet.

Aspect 39. A system comprising: a hydrodynamic separator element having an element inlet, a plurality of first element outlets, a second element outlet, and a plurality of curved microfluidic channels between the element inlet and the first element outlets, wherein each curved microfluidic channel has an inner wall and an outer wall and the hydrodynamic separator element is configured to concentrate particles along the inner wall; and a tangential flow filter having a feed inlet downstream of the plurality of first element outlets, a retentate outlet, a permeate outlet, and a filter media disposed between the feed inlet and the permeate outlet.

Aspect 40. The system of any one of aspects 39 and 41-59, further comprising a cell culture tank positioned downstream of the tangential flow filter.

Aspect 41. The system of any one of aspects 39-40 and 42-59, wherein the hydrodynamic separator element comprises a substrate layer defining a layer inlet, a plurality of first layer outlets and a second layer outlet, wherein the layer inlet is downstream of the element inlet, each first layer outlet is upstream of a first element outlet of the plurality of first element outlets, and the second layer outlet is upstream of the second element outlet, and wherein each of the plurality of curved microfluidic channels extend between the layer inlet and a first layer outlet of the plurality of first layer outlets.

Aspect 42. The system of any one of aspects 39-41 and 43-59, further comprising a permeate draining layer abutting the substrate layer, wherein the permeate draining layer is downstream of the first layer outlets.

Aspect 43. The system of any one of aspects 39-42 and 44-59, wherein the permeate draining layer extends laterally across the substrate layer.

Aspect 44. The system of any one of aspects 39-43 and 45-59, wherein the hydrodynamic separator element comprises a plurality of substrate layers in a stacked configuration, wherein each substrate layer defines a layer inlet, a plurality of first layer outlets and a second layer outlet, and a curved microfluidic channel extending between the layer inlet and a first layer outlet of the plurality of first layer outlets, wherein each layer inlet is downstream of the element inlet, each first layer outlet is upstream of a first element outlet, and each second layer outlet is upstream of the second element outlet.

Aspect 45. The system of any one of aspects 39-44 and 46-59, further comprising a permeate draining layer abutting each substrate layer, wherein the permeate draining layer is downstream of a plurality of first layer outlets.

Aspect 46. The system of any one of aspects 39-45 and 47-59, wherein each permeate draining layer extends laterally across a corresponding substrate layer.

Aspect 47. The system of any one of aspects 39-46 and 48-59, wherein the permeate draining layer is defined by a mesh material.

Aspect 48. The system of any one of aspects 39-47 and 49-59, wherein the permeate draining layer is a material layer defining a microchannel downstream of the plurality of first layer outlets.

Aspect 49. The system of any one of aspects 39-48 and 50-59 wherein each substrate layer defines a plurality of curved microfluidic channels each extending between the layer inlet and a first layer outlet of the plurality of first layer outlets.

Aspect 50. The system of any one of aspects 39-49 and 51-59, wherein each of the plurality of microfluidic channels is nested with an adjacent microfluidic channel.

Aspect 51. The system of any one of aspects 39-50 and 52-59, wherein each substrate layer comprises at least 6 microfluidic channels.

Aspect 52. The system of any one of aspects 39-51 and 53-59, wherein each microfluidic channel is within 12 mm, 10 mm, 5 mm, or 3 mm of an adjacent microfluidic channel within each substrate layer.

Aspect 53. The system of any one of aspects 39-52 and 54-59, wherein each microfluidic channel is within 12 mm, 10 mm, 5 mm, or 3 mm of an adjacent microfluidic channel within each substrate layer.

Aspect 54. The system of any one of aspects 39-53 and 55-59, wherein each microfluidic channel defines two curves in opposite directions.

Aspect 55. The system of any one of aspects 39-54 and 56-59, wherein the length of each of the plurality of curved microfluidic channels defines an arc of less than or equal to 270°.

Aspect 56. The system of any one of aspects 39-55 and 57-59, wherein the length of each of the plurality of curved microfluidic channels defines an arc of less than or equal to 200°.

Aspect 57. The system of any one of aspects 39-56 and 58-59, wherein the hydrodynamic separator element is configured to separate particles having diameters ranging from 10-20 microns from a liquid stream.

Aspect 58. The system of any one of aspects 39-57 and 59, wherein the hydrodynamic separator element is configured to separate one or both of mammalian cells and insect cells in a liquid stream.

Aspect 59. The system of any one of aspects 39-58, wherein the hydrodynamic separator element is configured to separate one or both of fish cells and avian cells in a liquid stream.

It should also be noted that, as used in this specification and the appended claims, the phrase “configured” describes a system, apparatus, or other structure that is constructed to perform a particular task or adopt a particular configuration. The word “configured” can be used interchangeably with similar words such as “arranged”, “constructed”, “manufactured”, and the like.

All publications and patent applications in this specification are indicative of the level of ordinary skill in the art to which this technology pertains. All publications and patent applications are herein incorporated by reference to the same extent as if each individual publication or patent application was specifically and individually indicated by reference. In the event that any inconsistency exists between the disclosure of the present application and the disclosure(s) of any document incorporated herein by reference, the disclosure of the present application shall govern.

This application is intended to cover adaptations or variations of the present subject matter. It is to be understood that the above description is intended to be illustrative, and not restrictive, and the claims are not limited to the illustrative embodiments as set forth herein.

What is claimed is:

1. A hydrodynamic separator element comprising:
a substrate layer defining:
a layer inlet,
a plurality of first layer outlets and a second layer outlet,
an outlet flow path upstream of the second layer outlet, and
a plurality of curved microfluidic channels arranged to operate in parallel, wherein each microfluidic channel defines:
a channel inlet downstream of the layer inlet,
a first channel outlet upstream of a first layer outlet of the plurality of first layer outlets, and a second channel outlet upstream of the outlet flow path,
wherein the substrate layer is non-permeable, and wherein each of the plurality of microfluidic channels is nested with an adjacent microfluidic channel.

2. The hydrodynamic separator element of any one of claims 1 and 3-14, wherein the length of each of the plurality of curved microfluidic channels defines an arc of less than or equal to 200°.

3. The hydrodynamic separator element of any one of claims 1-2 and 4-14, wherein each microfluidic channel defines two curves in opposite directions.

4. The hydrodynamic separator element of any one of claims 1-3 and 5-14, wherein the plurality of curved microfluidic channels is at least 6 microfluidic channels.

5. The hydrodynamic separator element of any one of claims 1-4 and 6-14, further comprising a permeate draining layer abutting the substrate layer, wherein the permeate draining layer is downstream of the plurality of first layer outlets.

6. The hydrodynamic separator element of any one of claims 1-5 and 7-14, wherein the permeate draining layer extends laterally across the substrate layer.

7. The hydrodynamic separator element of any one of claims 1-6 and 8-14, wherein the permeate draining layer is a material layer defining a microchannel downstream of the plurality of first layer outlets.

8. The hydrodynamic separator element of any one of claims 1-7 and 9-14, wherein the permeate draining layer is defined by a mesh material.

9. The hydrodynamic separator element of any one of claims 1-8 and 10-14, wherein the hydrodynamic separator element is a component of a cell retention device of a perfusion bioreactor.

10. The hydrodynamic separator element of any one of claims 1-9 and 11-14, wherein each microfluidic channel has an inner wall and an outer wall and each microfluidic channel is configured to concentrate particles in a liquid stream towards the inner wall.

11. The hydrodynamic separator element of any one of claims 1-10 and 12-14, wherein the hydrodynamic separator element is configured to separate particles ranging from 10-20 microns from a liquid stream.

12. The hydrodynamic separator element of any one of claims 1-11 and 13-14, wherein the hydrodynamic separator element is configured to separate one or both of mammalian cells and insect cells in a liquid stream.

13. The hydrodynamic separator element of any one of claims 1-12 and 14, further comprising:
a plurality of the substrate layers in a stacked configuration; and
a system inlet in direct fluid communication with each layer inlet, wherein the plurality of substrate layers are arranged to operate in parallel.

14. The hydrodynamic separator element of claim 13, further comprising a permeate draining layer abutting each substrate layer, wherein each permeate draining layer is downstream of a corresponding first layer outlet.

15. A separation assembly comprising:
a microfluidic separation element comprising a substrate layer, wherein the microfluidic separation element defines:
an element inlet,
a plurality of first element outlets defined by the substrate layer, and a second element outlet; and
a permeate draining layer abutting the substrate layer, wherein the permeate draining layer is downstream of the plurality of first element outlets.

16. The separation assembly of any one of claims 15 and 17-20, wherein the permeate draining layer extends laterally across the substrate layer.

17. The separation assembly of any one of claims 15-16 and 18-20, further comprising:
a first microfluidic channel defining a channel inlet downstream of the element inlet and
a channel outlet having a first channel outlet upstream of a first element outlet of the plurality of first element outlets and a second channel outlet upstream of the second element outlet.

18. The separation assembly of any one of claims 15-17 and 19-20, further comprising a plurality of microfluidic channels including the first microfluidic channel, wherein each microfluidic channel defines a channel inlet downstream of the element inlet and a first channel outlet upstream of a first element outlet of the plurality of first element outlets and a second channel outlet upstream of the second element outlet.

19. The separation assembly of any one of claims 15-18 and 20, wherein each of the plurality of microfluidic channels is nested with an adjacent microfluidic channel.

20. A system comprising:
a plurality of separation assemblies of any one of claims 15-19, wherein the separation assemblies are arranged in a stacked configuration; and
a system inlet in direct fluid communication with each element inlet, wherein the plurality of separation assemblies are arranged to operate in parallel.
